(19) United States
(12) Patent Application Publication
(10) Pub. No.: US 2021/0186394 A1
(43) Pub. Date: Jun. 24, 2021

(54) CATHETER EXTENSION SET AND RELATED METHODS

(71) Applicant: Becton, Dickinson and Company, Franklin Lakes, NJ (US)

(72) Inventors: Yiping Ma, Layton, UT (US); Jonathan Karl Burkholz, Salt Lake City, UT (US); Curtis H Blanchard, Riverton, UT (US); Bin Wang, Sandy, UT (US)

(21) Appl. No.: 17/114,705

(22) Filed: Dec. 8, 2020

Related U.S. Application Data
(60) Provisional application No. 62/951,736, filed on Dec. 20, 2019.

(51) Int. Cl. A61B 5/15

(52) U.S. Cl. CPC A61B 5/150992; A61B 5/150221; A61B 5/150625; A61B 5/150061

(57) ABSTRACT

A catheter extension set may include a distal end, which may include a luer adapter configured to couple to a catheter adapter. The catheter extension may also include a proximal end, which may include a blood collection device. The blood collection device may include a needle configured to receive a blood collection container. The catheter extension may include one or more extension tubes extending between the distal end and the proximal end. The catheter extension set may increase flow resistance, which may reduce a risk of hemolysis when there is a high-pressure differential within the catheter extension set. After a pressure differential within the catheter extension set is lowered, the catheter extension set may decrease flow resistance, which may facilitate more rapid blood collection.

### RELATED APPLICATIONS

This application claims the benefit of U.S. Provisional Application No. 62/951,736, filed Dec. 20, 2019, and entitled CATHETER EXTENSION SET AND RELATED METHODS, which is incorporated herein in its entirety.

### BACKGROUND

A catheter is commonly used to infuse fluids into vasculature of a patient. For example, the catheter may be used for infusing normal saline solution, various medicaments, or total parenteral nutrition. The catheter may also be used for withdrawing blood from the patient.

The catheter may include an over-the-needle peripheral intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

For blood withdrawal or collecting a blood sample from a patient, a blood collection container may be used. The blood collection container may include a syringe or a test tube with a rubber stopper at one end. In some instances, the blood collection container has had all or a portion of air removed from the test tube so pressure within the blood collection container is lower than ambient pressure. Such a blood collection container is often referred to as an internal vacuum or a vacuum tube. A commonly used blood collection container is a VACUTAINER® blood collection tube, available from Becton Dickinson & Company.

The blood collection container may be coupled to the catheter. When the blood collection container is coupled to the catheter, a pressure in the vein is higher than a pressure in the blood collection container, which pushes blood into the blood collection container, thus filling the blood collection container with blood. A vacuum within the blood collection container decreases as the blood collection container fills, until the pressure in the blood collection container equalizes with the pressure in the vein, and the flow of blood stops.

Unfortunately, as blood is drawn into the blood collection container, red blood cells are in a high shear stress state and susceptible to hemolysis due to a high initial pressure differential between the vein and the blood collection container. Hemolysis may result in rejection and discard of a blood sample. The high initial pressure differential can also result in catheter tip collapse, vein collapse, or other complications that prevent or restrict blood from filling the blood collection container. As the blood collection container fills, a pressure differential between the vein and the blood collection container decreases, and filling of the blood collection tube with blood slows significantly.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure generally relates to an extension set for a vascular access device, as well as related devices, systems, and methods. In some embodiments, a vascular access system may include the extension set coupled to the vascular access device. In some embodiments, the vascular access device may include a catheter, a needle, or another device suitable for blood collection.

In some embodiments, during blood collection, the extension set may facilitate adjustment of flow resistance within the vascular access system based on a pressure state within the vascular access system. In further detail, in some embodiments, as a pressure differential between a vein of a patient and a blood collection container coupled to the vascular access system changes during blood collection, the extension set may be responsive and may facilitate adjustment of flow resistance within the vascular access system. In some embodiments, the extension set may decrease a risk of hemolysis. In some embodiments, the extension set may simultaneously decrease the risk of hemolysis and decrease blood collection time during a process of blood withdrawal. In some embodiments, the extension set may also reduce a risk of collapse of the vein and/or the vascular access device.

In some embodiments, in response to the pressure differential being high, such as when the blood collection container is first coupled to the vascular access system, the extension set may facilitate increased flow resistance within the vascular access system to distribute the pressure differential and reduce shear stress experienced by red blood cells. In some embodiments, as the blood collection container fills with blood, a vacuum within a fluid pathway of the vascular access system may decrease, and the pressure differential may decrease. Thus, in some embodiments, in order to shorten the blood collection time, the extension set may facilitate decreased flow resistance within the vascular access system while still maintaining the shear stress of the blood cells below a threshold level.

In some embodiments, the extension set may be configured to couple to a catheter assembly and may be referred to as a catheter extension set. In some embodiments, the extension set may include a distal end, which may include a luer adapter configured to couple to a catheter adapter or another suitable vascular access device. In some embodiments, the extension set may include a proximal end, which may include a blood collection device. In some embodiments, the blood collection device may include or correspond to a blood collection container. In some embodiments, the blood collection container may include a syringe, an evacuated blood collection tube, a small sample collection device, or any other container configured to collect blood from a patient via a pressure differential. In some embodiments, the blood collection device may include a needle assembly, which may include a needle configured to receive the blood collection container. In these and other embodiments, the blood collection container may include the evacuated blood collection tube.

In some embodiments, the extension set may include an extension tube, which may extend between the distal end of the extension set and the proximal end of the extension set. In some embodiments, the extension set may include a clamp, which may be disposed on the extension tube. In some embodiments, the clamp may be configured to move between a clamped position and an unclamped position. In some embodiments, the clamp may prevent or reduce fluid flow through the extension tube in response to the clamp being in the clamped position.

In some embodiments, the luer adapter may be a first luer adapter. In some embodiments, the blood collection device may include a second luer adapter. In some embodiments, the extension set may include a third luer adapter, which may be coupled to the second luer adapter. In some embodiments, the extension tube may include a distal end and a proximal end. In some embodiments, the distal end of the extension tube may be integrated with the first luer adapter. In some embodiments, the proximal end of the extension tube may be integrated with the third luer adapter. In other embodiments, the proximal end of the extension tube may be integrated with the blood collection device.

In some embodiments, the extension tube may include an absorbent material or an indicator tube. In some embodiments, an inner surface of the extension tube may include an absorbent material or an indicator tube. In some embodiments, the absorbent material and/or the indicator tube may include one or more markers. In some embodiments, blood within the extension tube may be configured to flow proximate the absorbent material or through the indicator tube. In some embodiments, in response to blood reaching or saturating a particular marker, a clinician may change the flow resistance within the vascular access system via the extension set. In some embodiments, to decrease the flow resistance within the vascular access system, the clinician may remove the extension set from the vascular access system, move the clamp to the unclamped position, or perform another action to decrease the flow resistance as may be further described in the present disclosure.

In some embodiments, the extension set may include one or more extension tubes. In some embodiments, the extension tube may be a first extension tube. In some embodiments, the extension set may include a second extension tube. In some embodiments, a flow resistance of the first extension tube may be greater than a flow resistance of the second extension tube. For example, the first extension tube may be longer than the second extension tube. Additionally or alternatively, in some embodiments, an inner diameter of the first extension tube may be less than an inner diameter of the second extension tube. In some embodiments, the flow resistance of the first extension tube may be equal to the flow resistance of the second extension tube. In some embodiments, the second extension tube may include any combination of length and inner diameter such that the flow resistance of the second extension tube is the same or less than the flow resistance of the first extension tube.

In some embodiments, the first extension tube and the second extension tube may extend between the distal end of the extension set and the proximal end of the extension set. In some embodiments, the clamp may be disposed on the second extension tube. In some embodiments, the second extension tube may extend through the clamp. In some embodiments, the second extension tube may include the absorbent material or the indicator tube. In some embodiments, blood within the second extension tube may be configured to flow proximate the absorbent material or through the indicator tube.

In some embodiments, the first extension tube may be disposed within the second extension tube. In some embodiments, in response to the clamp being in the clamped position, blood may flow through the first extension tube, and blood flow between an outer surface of the first extension tube and an inner surface of the second extension tube may decrease or stop. In some embodiments, to decrease or stop the flow resistance within the vascular access system after the blood collection container is partially filled and the pressure differential has decreased, the clinician may move the clamp to the unclamped position to increase a flow rate given the pressure differential has decreased.

In some embodiments, the second extension tube may include a distal end and a proximal end. In some embodiments, the distal end of the first extension tube and the distal end of the second extension tube may be integrated with the first luer adapter. In some embodiments, the proximal end of the first extension tube and the proximal end of the second extension tube may be integrated with the third luer adapter.

In some embodiments, in response to the second luer adapter being tightened with respect to the third luer adapter, a first fluid pathway through the first extension tube may be open, and a second fluid pathway through the second extension tube may be closed. In some embodiments, in response to the second luer adapter being loosened with respect to the third luer adapter, the first fluid pathway through the first extension tube may be open, and the second fluid pathway through the second extension tube may be open. In some embodiments, to decrease the flow resistance within the vascular access system after the blood collection container is partially filled and the pressure differential has decreased, the clinician may loosen the second luer adapter with respect to the third luer adapter or move the second luer adapter from a tightened position to a loosened position. As a result, in these and other embodiments, a flow rate may increase.

In some embodiments, the extension set may include a pressure-sensitive valve within the extension set. In some embodiments, the pressure-sensitive valve may be disposed in the second extension tube. In some embodiments, the pressure-sensitive valve may be disposed in a portion of the fluid pathway of the extension set distal to the first extension tube and the second extension tube. In some embodiments, the fluid pathway of the extension set may extend through the proximal end of the extension set and the distal end of the extension set. In some embodiments, the fluid pathway of the extension set may include the first fluid pathway of the first extension tube and/or the second fluid pathway of the second extension tube. In some embodiments, the pressure-sensitive valve may be disposed at a junction of the first fluid pathway and the second fluid pathway.

In some embodiments, in response to a first predetermined pressure differential within the extension set, the pressure-sensitive valve may be open with respect to the first extension tube and closed with respect to the second extension tube. In some embodiments, in response to a second predetermined pressure differential within the extension set, the pressure-sensitive valve may be closed with respect to the first extension tube and open with respect to the second extension tube. In some embodiments, the first predetermined pressure differential may be greater than the second predetermined pressure differential. In some embodiments, the first predetermined pressure differential and the second predetermined pressure differential may correspond to the pressure differential between the vein of the patient and the blood collection container coupled to the vascular access system.

In some embodiments, a method of blood collection or collecting a blood sample may include inserting a catheter of a catheter system into vasculature of the patient. In some embodiments, the catheter system may include a catheter assembly, which may include a catheter adapter and the catheter. In some embodiments, the catheter may be secured within the catheter adapter and may extend distally from the catheter adapter.

In some embodiments, the method of blood collection may include coupling the extension set to the catheter adapter. In some embodiments, the method of blood collection may include blocking blood flow through the second extension tube. In some embodiments, after inserting the catheter of the catheter system into the vasculature of the patient, coupling the catheter extension set to the catheter adapter, and blocking blood flow through the second extension tube, the method of blood collection may include coupling a blood collection container to the blood collection device or the third luer adapter. In some embodiments, in response to coupling the blood collection container to the blood collection device or the third luer adapter, the blood collection container may begin to fill with blood. In some embodiments, in response to the blood collection container filling partially with blood, the method of blood collection may include opening the second extension tube such that increased blood flows through the second extension tube.

In some embodiments, the second extension tube may extend through the clamp. In these embodiments, blocking blood flow through the second extension tube may include moving the clamp to the clamped position, and opening the second extension tube may include moving the clamp to the unclamped position. In some embodiments, blocking blood flow through the second extension tube may include tightening the second luer with respect to the third luer adapter. In some embodiments, opening the second extension tube may include loosening the second luer with respect to the third luer adapter.

In some embodiments, the extension set may include a roller clamp. In some embodiments, the roller clamp may include a roller moveable within a track. In some embodiments, the first extension tube and the second extension tube may extend through the roller clamp generally perpendicular to the track. In some embodiments, when blood flow through the second extension tube is blocked, the roller may be disposed in a first position proximate the second extension tube. In some embodiments, opening the second extension tube may include moving the roller along the track away from the second extension tube and toward the first extension tube.

In some embodiments, the distal end of the extension set may include a three-way stopcock, which may include a first port, a second port, and a third port. In some embodiments, the first extension tube may be coupled with the first port, the second extension tube may be coupled to the second port, and the third port may include the first luer adapter configured to couple to the catheter adapter. In some embodiments, blocking blood flow through the second extension tube may include rotating the three-way stopcock to a first position. In some embodiments, opening the second extension tube may include rotating the three-way stopcock to a second position.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example extension set, according to some embodiments;

FIG. 1B is a cross-sectional view of the extension set of FIG. 1A, according to some embodiments;

FIG. 1C is an upper perspective view of the extension set of FIG. 1A coupled to an example catheter assembly, according to some embodiments;

FIG. 2A is an upper perspective view of another example extension set, according to some embodiments;

FIG. 2B is a cross-sectional view of the extension set of FIG. 2B, according to some embodiments;

FIG. 2C is an upper perspective view of the extension set of FIG. 2A coupled to the catheter assembly of FIG. 1C, according to some embodiments;

FIG. 3A is an upper perspective view of another example extension set, according to some embodiments;

FIG. 3B is a cross-sectional view of the extension set of FIG. 3A coupled to another example catheter assembly, according to some embodiments;

FIG. 3C is an upper perspective view of the extension set of FIG. 3A coupled to the catheter assembly of FIG. 3B, according to some embodiments;

FIG. 4A is an upper perspective view of another example extension set, according to some embodiments;

FIG. 4B is an upper perspective view of the extension set of FIG. 4A coupled to the catheter assembly of FIG. 3B, according to some embodiments;

FIG. 4C is a cross-sectional view of the extension set of FIG. 4A coupled to the catheter assembly of FIG. 3B, according to some embodiments;

FIG. 5A is a schematic diagram of the extension set of FIG. 3A, illustrating an example clamp in a clamped position prior to entry of blood into the extension set, according to some embodiments;

FIG. 5B is a schematic diagram of the extension set of FIG. 3A, illustrating blood flowing through an example first extension tube under a high initial pressure differential, according to some embodiments;

FIG. 5C is a schematic diagram of the extension set of FIG. 3A, illustrating the clamp removed or unclamped in response to a lower pressure differential, according to some embodiments;

FIG. 6A is a cross-sectional view of an example second luer adapter and an example third luer adapter prior to being coupled together, according to some embodiments;

FIG. 6B is a cross-sectional view of the second luer adapter and the third luer adapter of FIG. 6A, illustrating the second luer adapter tightened with respect to the third luer adapter, according to some embodiments;

FIG. 6C is a cross-sectional view of the second luer adapter and the third luer adapter of FIG. 6A, illustrating the second luer adapter loosened with respect to the third luer adapter, according to some embodiments;

FIG. 7A is an upper perspective view of an example roller clamp that may be used with the extension set of FIG. 3A or the extension set of FIG. 4A, according to some embodiments;

FIG. 7B is a cross-sectional view of the roller clamp of FIG. 7A, illustrating an example roller in an example first position, according to some embodiments;

FIG. 7C is a cross-sectional view of the roller clamp of FIG. 7B, illustrating the roller in an example second position, according to some embodiments;

FIG. 8 is a cross-sectional view of another example extension set coupled to the catheter assembly of FIG. 3B, according to some embodiments;

FIG. 9A is an upper perspective view of another example extension set coupled to the catheter assembly of FIG. 3B, according to some embodiments;

FIG. 9B is an enlarged upper perspective view of a portion of the extension set of FIG. 9A, according to some embodiments;

FIG. 9C is a cross-sectional view of the extension set of FIG. 9A, according to some embodiments;

FIG. 9D is a transverse cross-sectional view of the extension set of FIG. 9A, illustrating another example first extension tube disposed within an example second extension tube and the second extension tube clamped, according to some embodiments;

FIG. 9E is a transverse cross-sectional view of the extension set of FIG. 9A, illustrating the first extension tube disposed within the second extension tube, according to some embodiments;

FIG. 10A is a cross-sectional view of a portion of another example first extension tube, illustrating an example absorbent material prior to entry of blood into the first extension tube, according to some embodiments;

FIG. 10B is a cross-sectional view of the portion of the first extension tube of FIG. 10A, illustrating the absorbent material in response to blood flowing through the first extension tube under a high initial pressure differential, according to some embodiments;

FIG. 11A is a cross-sectional view of a portion of another example first extension tube, illustrating an example indicator tube prior to entry of blood into the first extension tube, according to some embodiments;

FIG. 11B is a cross-sectional view of the portion of the first extension tube of FIG. 11A, illustrating the indicator tube in response to blood flowing through the first extension tube under the high initial pressure differential, according to some embodiments;

FIG. 12A is a cross-sectional view of an example junction between another example first extension tube and an example second extension tube, illustrating the junction prior to entry of blood into the junction, according to some embodiments;

FIG. 12B is a cross-sectional view of the junction of FIG. 12A, illustrating the junction in response to blood flowing through the junction under the high initial pressure differential, according to some embodiments;

FIG. 12C is a cross-sectional view of the junction of FIG. 12A, illustrating the junction in response to blood flowing through the junction under a lower pressure differential compared to the high initial pressure differential, according to some embodiments;

FIG. 13 is a partial cutaway view of another example extension set coupled to the catheter assembly of FIG. 3B, according to some embodiments;

FIG. 14A is an upper perspective view of an example catheter system, according to some embodiments; and

FIG. 14B is an upper perspective view of another example catheter system, according to some embodiments.

### DESCRIPTION OF EMBODIMENTS

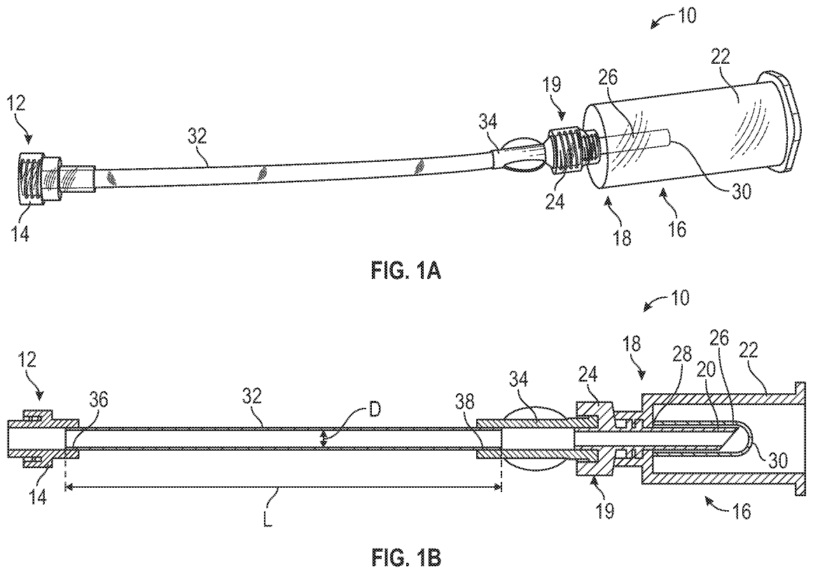

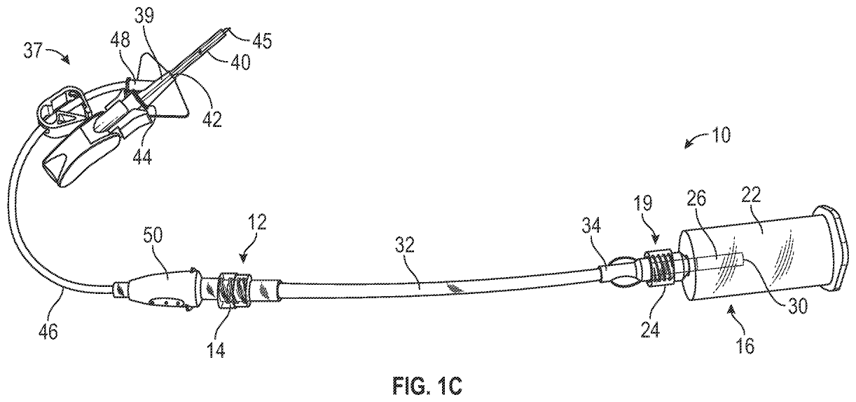

Referring now to FIGS. 1A-1C, in some embodiments, an extension set 10 may include a distal end 12, which may include a luer adapter 14 configured to couple to a catheter adapter or another suitable vascular access device. In some embodiments, the extension set 10 may include a proximal end 16, which may include a blood collection device 18. In some embodiments, the blood collection device 18 may include or correspond to a blood collection container. In some embodiments, the blood collection container may include a syringe, an evacuated blood collection tube, a small sample collection device, or any other container configured to collect blood from a patient via a pressure differential.

In some embodiments, the blood collection device 18 may include a needle assembly 19, which may include a needle 20 configured to receive a blood collection container. In these and other embodiments, the blood collection container may include the evacuated blood collection tube. In some embodiments, the blood collection container has all or a portion of air removed so pressure within the blood collection container is lower than ambient pressure.

In some embodiments, the needle assembly 19 may include one or more threads, which may be configured to couple to a holder 22, which may be generally cylindrical and may be configured to hold the blood collection container. In some embodiments, the holder 22 may be integrally formed with the needle assembly 19 or coupled to the needle assembly 19 via bonding or another suitable method. In some embodiments, the holder 22 may surround the needle 20. In some embodiments, the needle assembly 19 and the holder 22 may include or correspond to a luer lock access device, such as, for example, the VACUTAINER® LUER-LOK™ ACCESS DEVICE available from Becton Dickinson & Company. In some embodiments, the holder 22 may include or correspond to the blood collection tube holder 127 described in U.S. Patent Application No. 62/928,69, filed Oct. 30, 2019, entitled “BLOOD COLLECTION SYSTEM WITH USER-ADJUSTED PRESSURE MANAGEMENT AND RELATED METHODS,” which is incorporated by reference in its entirety.

In some embodiments, the luer adapter 14 may be a first luer adapter 14. In some embodiments, the extension set 10 may include a second luer adapter 24. In some embodiments, the blood collection device 18 may include the second luer adapter 24. In some embodiments, the needle 20 may be integrated with the second luer adapter 24. In some embodiments, a proximal end of the needle 20 may be enveloped within an elastomeric sheath 26. In some embodiments, the elastomeric sheath 26 may include an open distal end 28 and a closed proximal end 30. In some embodiments, in response to the blood collection container pushing the elastomeric sheath 26 distally, the needle 20 may pierce the elastomeric sheath 26, and the needle 20 may insert into a cavity of the blood collection container.

In some embodiments, the extension set 10 may include an extension tube 32, which may extend between the distal end 12 of the extension set 10 and the proximal end 16 of the extension set 10. In some embodiments, the extension tube 32 may be rigid or semi-rigid, which may reduce a likelihood of kinking.

In some embodiments, the extension set 10 may include a third luer adapter 34, which may be coupled to the second luer adapter 24. In some embodiments, the extension tube 32 may include a distal end 36 and a proximal end 38. In some embodiments, the distal end 36 may be coupled to or integrated with the first luer adapter 14. In some embodiments, the proximal end of the extension tube 32 may be coupled to or integrated with the third luer adapter 34. In other embodiments, the proximal end of the extension tube 32 may be coupled to or integrated with the blood collection device 18.

In some embodiments, the extension set 10 may act as a flow resistor in a fluid pathway of a catheter system, illustrated, for example, in FIG. 1C, or another vascular access system. In some embodiments, the catheter system may include a catheter assembly 37, which may include a catheter adapter 39 and a catheter 40. In some embodiments, the catheter 40 may be secured within the catheter adapter 39 and may extend distally from the catheter adapter 39. In some embodiments, the catheter adapter 39 may include a distal end 42, a proximal end 44, and a lumen extending through the distal end 42 and the proximal end 44. In some embodiments, an introducer needle 45 may extend from a needle shield through the catheter 40.

In some embodiments, the extension set 10 may be coupled to the catheter assembly 37 in any number of suitable ways. In some embodiments, the catheter assembly 37 may be integrated. In further detail, in some embodiments, another extension tube 46 may extend from a side port 48 the catheter adapter 39. In some embodiments, a proximal end of the other extension tube 46 may include a fourth luer adapter 50, which may be coupled to the first luer adapter 14. In some embodiments, the catheter assembly 37 may be straight and/or the first luer adapter 14 may be coupled to the proximal end 44 of the catheter adapter 39. In some embodiments, one or more of the first luer adapter 14, the second luer adapter 24, the third luer adapter 34, and the fourth luer adapter 50 may include a slip or thread or clip male luer adapter, a slip or thread female luer adapter, a needleless connector, a blunt cannula, or another suitable access device.

In some embodiments, the catheter assembly 37 may include or correspond to any suitable catheter assembly, such as, for example, the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable catheter assembly. In some embodiments, the catheter 40 may include a peripheral intravenous catheter (PIVC), a peripherally inserted central catheter (PICC), or a midline catheter.

In some embodiments, the fluid pathway of the catheter system may include one or more of the following: the catheter 40, the catheter adapter 39, the other extension tube 46, the fourth luer adapter 50, the first luer adapter 14, the extension tube 32, the third luer adapter 34, the second luer adapter 24, and the blood collection device 18. In some embodiments, the extension set 10 may lower a flow rate of blood within the fluid pathway of the catheter system, which may in turn lower a shear rate for hemolysis management. In some embodiments, the catheter assembly 37 may be substituted with another type of vascular access device, such as, for example, a venipuncture device, an infusion disposable, a blood collection access device, or a blood collection container.

Blood cell experiences shear stress as it flows in a fluid pathway. The maximum shear stress is along the wall of the fluid pathway, or wall shear stress. Wall shear stress on blood cells is considered a major source of mechanical damage to blood cells. For a cylindrical fluid path, the wall shear stress is typically expressed as:

$$τ = \frac{1}{2} \cdot \frac{Δ p}{L} \cdot ( kr )$$

in which [symbol] P is the pressure drop along a path with a length of L and an interior radius of r. k is shrinkage index.

To fill a certain volume of collection tube, V, with a flow rate of Q, the time needed can be simply assessed by:

$$t = \frac{v}{ℚ} = 8 µ v \cdot \frac{1}{π {r}^{4}} / ( \frac{Δ p}{L} )$$

in which μ is the dynamic viscosity of the fluid. Hemolysis is typically associated with both the wall shear stress and the time a blood cell is exposed to wall shear stress. From literature, it has been widely considered that hemolysis index can be approached as a function of:

HI (%)=A*t^{α}*τ^{β}

in which A, α, and β are coefficients.

In principle, the hemolysis index is related to pressure gradient and cross-sectional characteristic dimension:

$$HI ( \% ) ∝ {( \frac{Δ P}{l} )}^{β - α} \cdot {( \frac{1}{r} )}^{4 a - β}$$

In some embodiments, a length of the extension tube 32 may be selected based on one or more of the following: a gauge and/or length of a particular catheter, a particular catheter assembly configuration, or a clinical setup. In some embodiments, the extension tube 32 may include a length L from the first luer adapter 14 to the second luer adapter 24. In some embodiments, the extension tube 32 may include an inner diameter D.

Fluid flow in an extension tube with a tubular fluid pathway therethrough can be analyzed using Poiseuille's equation:

$$Q = \frac{π {D}^{4} Δ P}{128 μ L} = \frac{Δ P}{{R}_{f}}$$

where ΔP is a change in pressure gradient across the length of the extension tube, D and L are the inner diameter and length, respectively, of the tubular fluid pathway through the extension tube, μ is the viscosity of a fluid, and

$${R}_{f} = \frac{128 μ L}{π {D}^{4}}$$

is the fluid resistance. The extension tube may include or correspond to the extension tube 32. Since μ is the viscosity of the fluid and not part of the extension tube geometry, a geometric factor G_{f} is defined such that R_{f} (the fluid resistance) is

$${R}_{f} = \frac{128 μ}{π} {G}_{f} , {G}_{f} = \frac{L}{{D}^{4}} .$$

In some embodiments, the extension tube 32 may have multiple sections with lengths (L1, L2, L3) and inner diameters of (D1, D2, D3), the geometric factor is then:

$${G}_{f} = \frac{L 1}{D {1}^{4}} + \frac{L 2}{D {2}^{4}} + \frac{L 3}{D {3}^{4}}$$

In some embodiments, the extension tube 32 may have an inner diameter that changes over the length of the extension tube, the geometric factor is then:

$${G}_{f} = {\int }_{0}^{L} \frac{d l}{{D ( l )}^{4}}$$

In some embodiments, the extension tube 32 may have a cross section that is not circular. In this case, the geometric factor can be determined by measuring the flow rate (Q) at given pressure (ΔP) with known viscosity (μ) fluid:

$${G}_{f} = \frac{π Δ P}{128 μ Q}$$

The G_{f} value may be selected to reduce the max shear stress for each catheter gauge to be the same or less than the max shear stress of a 21G VACUTAINER® UTLRATOUCH™ push button blood collection set (available from Becton Dickinson & Company), which was previously considered the gold standard for blood draws. In some embodiments, G_{f} may be equal to or more than 3.83E+06 (l/in³) when a 18G catheter is used, which may reduce the wall sheer stress to reduce hemolysis. In some embodiments, G_{f} may be equal to or more than 3.27E+06 (l/in³) when a 20G catheter is used, which may reduce the wall sheer stress to reduce hemolysis. In some embodiments, G_{f} may be equal to or more than 3.33E+06 (l/in³) when a 22G catheter is used, which may reduce the wall sheer stress to reduce hemolysis. In some embodiments, G_{f} may be equal to or more than 1.50E+07 (l/in³) when a 24G catheter is used, which may reduce the wall sheer stress to reduce hemolysis. In some embodiments, G_{f} may include another value. In some embodiments, G_{f} value may be selected to reduce the max shear stress for each catheter gauge to be the same or less than the max shear stress of a 25G VACUTAINER® ULTRATOUCH™ push button blood collection set (available from Becton Dickinson & Company).

In some embodiments, when a 18G catheter is used, G_{f} may be equal to 3.83E+06 (l/in³) plus or minus 10 percent, plus or minus 25 percent, plus or minus 50 percent, or plus or minus 75 percent, which may reduce the wall sheer stress to reduce hemolysis. In some embodiments, when a 20G catheter is used, G_{f} may be equal to 3.27E+06 (l/in³) plus or minus 10 percent, plus or minus 25 percent, plus or minus 50 percent, or plus or minus 75 percent, which may reduce the wall sheer stress to reduce hemolysis. In some embodiments, when a 22G catheter is used, G_{f} may be equal to 3.33E+06 (l/in³) plus or minus 10 percent, plus or minus 25 percent, plus or minus 50 percent, or plus or minus 75 percent, which may reduce the wall sheer stress to reduce hemolysis. In some embodiments, when a 24G catheter is used, G_{f} may be equal to 1.50E+07 (l/in³) plus or minus 10 percent, plus or minus 25 percent, plus or minus 50 percent, or plus or minus 75 percent, which may reduce the wall sheer stress to reduce hemolysis. In some embodiments, G_{f} may include another value, which may be selected based on a gauge of the catheter. In some embodiments, G_{f} values may be selected to be the same for 22G through 18G catheters.

In some embodiments, the fluid pathway of the catheter system, which may include one or more of the needle assembly 19, the extension tube 32, and the catheter assembly 37 (which may include the other extension tube 46), may include an entirety of a blood collection pathway through which blood flows during blood collection. The system geometric factor G_{fs} for the fluid pathway of the catheter system can be determined in similar fashion as described earlier. In some embodiments, the system geometric factor G_{fs} may be equal to or more than 7.34E+06 (l/in³). In some embodiments, G_{fs} may include another value. In some embodiments, the system geometric factor G_{fs} may be 7.34E+06 (l/in³) plus or minus 10 percent, plus or minus 25 percent, plus or minus 50 percent, or plus or minus 75 percent. In some embodiments, G_{fs} may include another value, which may be selected based on a gauge of the catheter.

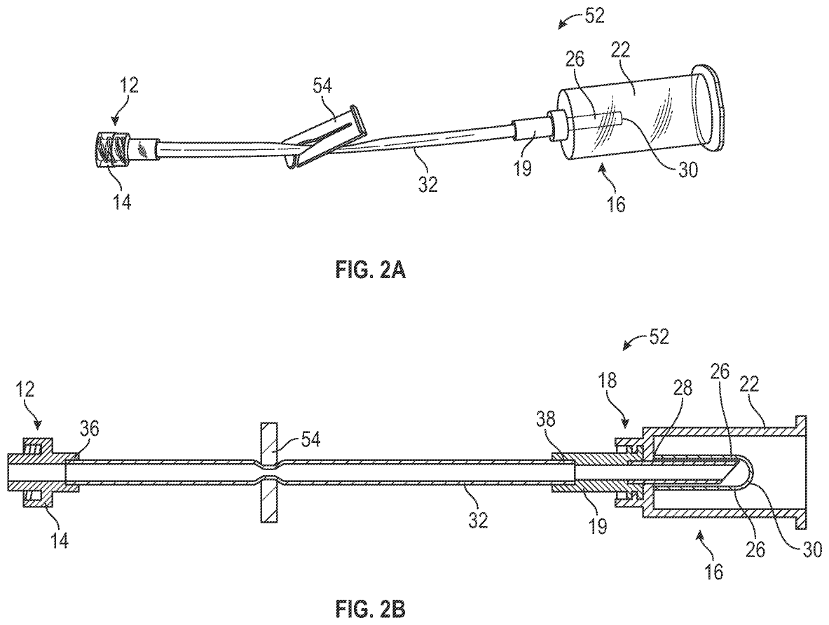

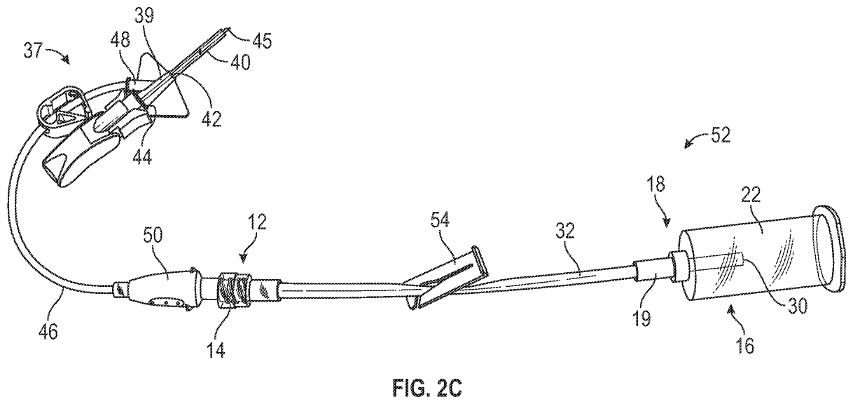

Referring now to FIGS. 2A-2C, an extension set 52 is illustrated, according to some embodiments. In some embodiments, the extension set 52 may be similar or identical to the extension set 10 of FIGS. 1A-1C in terms of one or more included features and/or operation. In some embodiments, the proximal end of the extension tube 32 may be integrated with the blood collection device 18. In some embodiments, the extension set 52 may include a clamp 54, which may be disposed on the extension tube 32. In some embodiments, the clamp 54 may be configured to move between a clamped position and an unclamped position or between a more clamped position and a less clamped position. In some embodiments, the clamp 54 may prevent or reduce fluid flow through the extension tube 32 in response to the clamp 54 being in the clamped position.

In some embodiments, the clinician may adjust flow resistance within the catheter system by manually changing fluid characteristics of the catheter system via the clamp 54. In some embodiments, in response to the clamp 54 being in the clamped position, flow resistance within the catheter system may be increased and blood flow through the extension tube 32 may be reduced. In these embodiments, a risk of hemolysis may be reduced. In some embodiments, to decrease the flow resistance within the catheter system after the blood collection container nears filling, the clinician may move the clamp to the unclamped position, which may allow faster blood collection when a risk of hemolysis is reduced.

In some embodiments, the clamp 54 may include a slide clamp, which may include a slot that becomes progressively narrower. In these and other embodiments, the extension tube 32 may be flexible and compliant. In some embodiments, the clinician may adjust an inner diameter of the extension tube 32 by adjusting a depth of the extension tube 32 within the slot of the slide clamp. The clinician may in turn adjust a flow resistance within the extension set 52. In some embodiments, the clamp 54 may include a roller clamp, a slide clamp, a pinch clamp, or another suitable type of clamp.

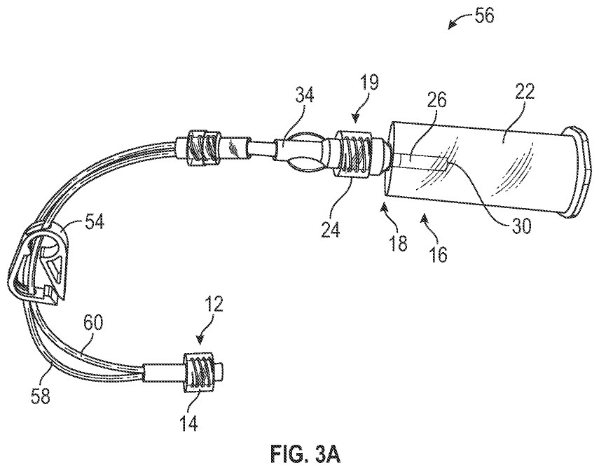

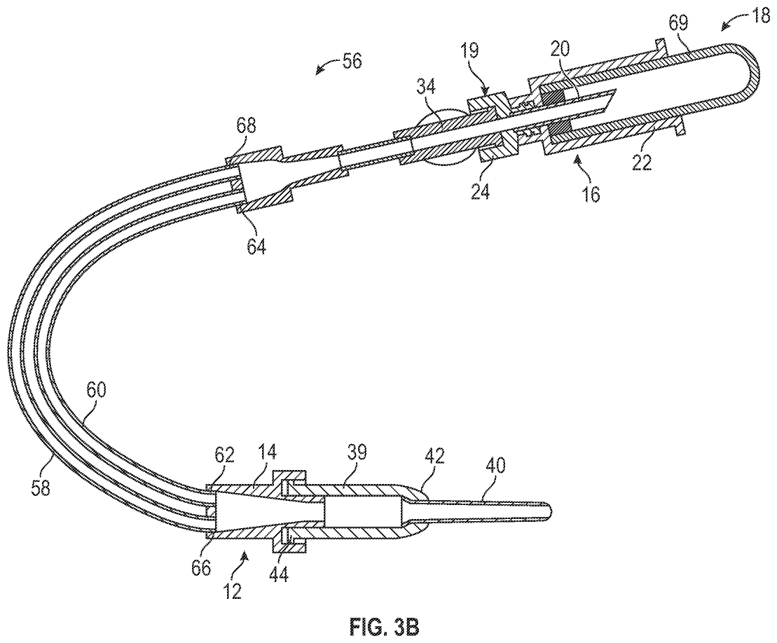

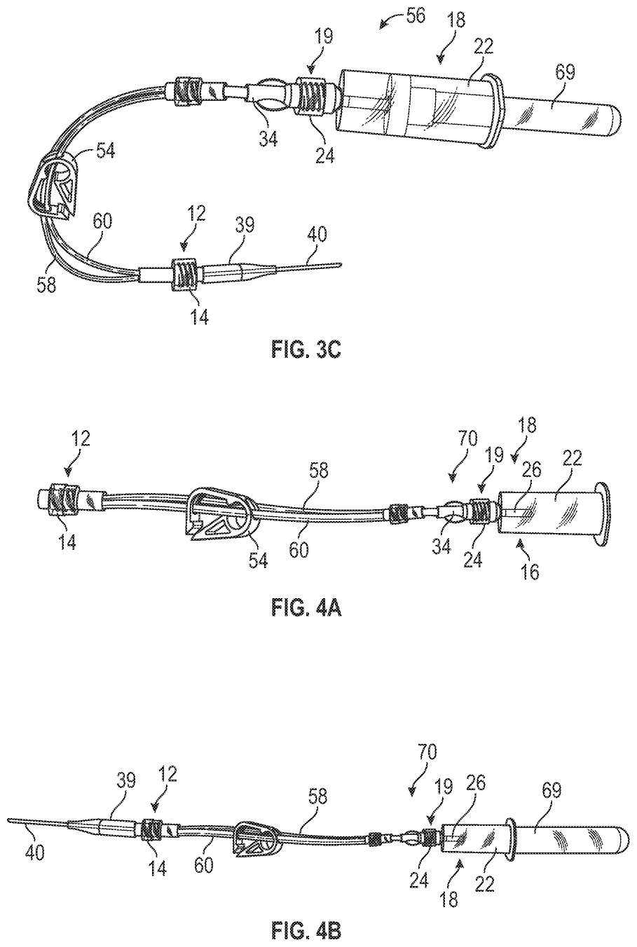

Referring now to FIGS. 3A-3C, an extension set 56 is illustrated, according to some embodiments. In some embodiments, the extension set 56 may be similar or identical to the extension set 10 of FIGS. 1A-1C and/or the extension set 52 in terms of one or more included features and/or operation. In some embodiments, the extension set 56 may include one or more extension tubes. In some embodiments, the extension tube 32 may include or correspond to a first extension tube 58 of the extension set 56. In some embodiments, the extension set 56 may include a second extension tube 60.

It is understood that in some embodiments, any suitable first lumen may be substituted for the first extension tube 58 and/or any suitable second lumen may be substituted for the second extension tube 60. Thus, in some embodiments, a particular extension set may not include the first extension tube 58 and/or the second extension tube 60 and may instead include the first lumen and/or the second lumen. In some embodiments, the first lumen and the second lumen may be disposed in a single multi-lumen extension tube or any other suitable structure. In some embodiments, a distal end of the single multi-lumen extension tube may be coupled to or integrated with the first luer adapter 14. In some embodiments, a proximal end of the single multi-lumen extension tube may be coupled to or integrated with the third luer adapter 34. In some embodiments, a proximal end of the single multi-lumen extension tube may be coupled to or integrated with the blood collection device 18.

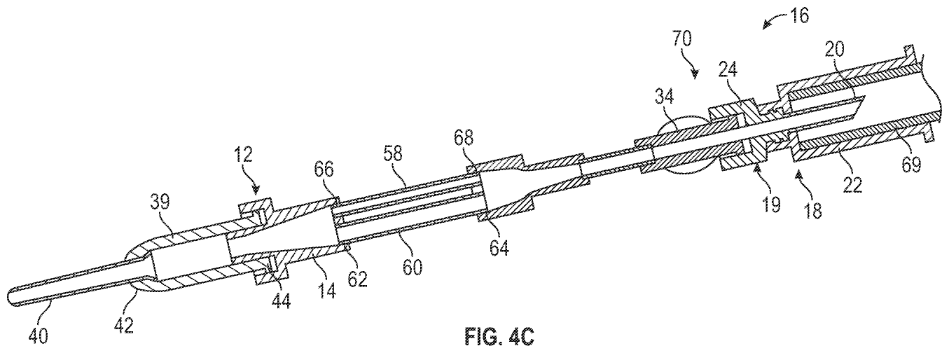

In some embodiments, a flow resistance of the first extension tube 58 may be greater than a flow resistance of the second extension tube 60. For example, the first extension tube 58 may be longer than the second extension tube 60, as illustrated, for example, in FIGS. 3A-3C. Additionally or alternatively, in some embodiments, an inner diameter of the first extension tube 58 may be less than an inner diameter of the second extension tube 60, as illustrated, for example, in FIGS. 4A-4C. In some embodiments, the flow resistance of the first extension tube 58 may be equal to the flow resistance of the second extension tube 60. In some embodiments, the second extension tube 60 may include any combination of length and inner diameter such that the flow resistance of the second extension tube 60 is the same or less than the flow resistance of the first extension tube 58.

In some embodiments, the first extension tube 58 and the second extension tube 60 may extend between a distal end 12 of the extension set 56 and a proximal end 16 of the extension set 56. In some embodiments, the proximal end 16 of the extension set may include the blood collection device 18 or the third luer connector 34. In some embodiments, a clamp 54 may be disposed on the second extension tube 60. In some embodiments, the second extension tube 60 may extend through the clamp 54.

In some embodiments, the second extension tube 60 may include a distal end 62 and a proximal end 64. In some embodiments, a distal end 66 of the first extension tube 58 and the distal end 62 of the second extension tube 60 may be integrated with the first luer adapter 14, as illustrated, for example, in FIG. 3B. In some embodiments, a proximal end 68 of the first extension tube 58 and the proximal end 64 of the second extension tube 60 may be integrated with the third luer adapter 34 or there may be one or more components between the proximal ends 64, 68 and the third luer adapter 34, such as, for example, a short tubing. In some embodiments, the proximal end 68 of the first extension tube 58 and the proximal end 64 of the second extension tube 60 may be integrated with the blood collection device 18.

In some embodiments, in response to the clamp 54 being in the clamped position, flow resistance may be increased as blood may flow through the first extension tube 58. In some embodiments, in response to the clamp 54 being in the clamped position, blood flow through the second extension tube 60 may be reduced or nothing. In some embodiments, to decrease the flow resistance after the blood collection container is partially filled and the pressure differential has decreased, the clinician may move the clamp 54 to the unclamped position, which may allow faster blood collection. An example blood collection container 69 is illustrated in FIGS. 3B-3C.

Referring now to FIGS. 4A-4C, an extension set 70 is illustrated, according to some embodiments. In some embodiments, the extension set 70 may be similar or identical to one or more of the following in terms of one or more included features and/or operation: the extension set 10 of FIGS. 1A-1C, the extension set 52 of FIGS. 2A-2C, and the extension set 56 of FIGS. 3A-3C. In some embodiments, an inner diameter of the first extension tube 58 may be less than an inner diameter of the second extension tube 60. In some embodiments, the first extension tube 58 and the second extension tube 60 may be a same length.

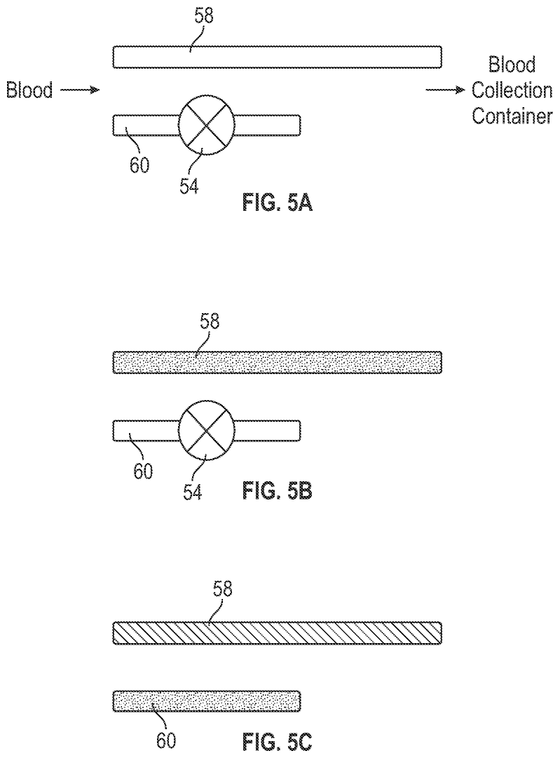

Referring now to FIGS. 5A-5C, a schematic diagram of an extension set having multiple extension tubes is illustrated, according to some embodiments. The extension set may correspond to the extension set 56 or the extension set 70, for example. In some embodiments, as illustrated in FIG. 5A, the clamp 54 may be in the clamped position prior to entry of blood into the extension set. In some embodiments, as illustrated in FIG. 5B, blood may flow through the first extension tube 58 in response to the second extension tube 60 being clamped. In some embodiments, as illustrated in FIG. 5C, in response to the clamp being removed, unclamped, or less clamped, increased blood may flow through the second extension tube 60, which may increase a total amount of blood flowing through the extension set.

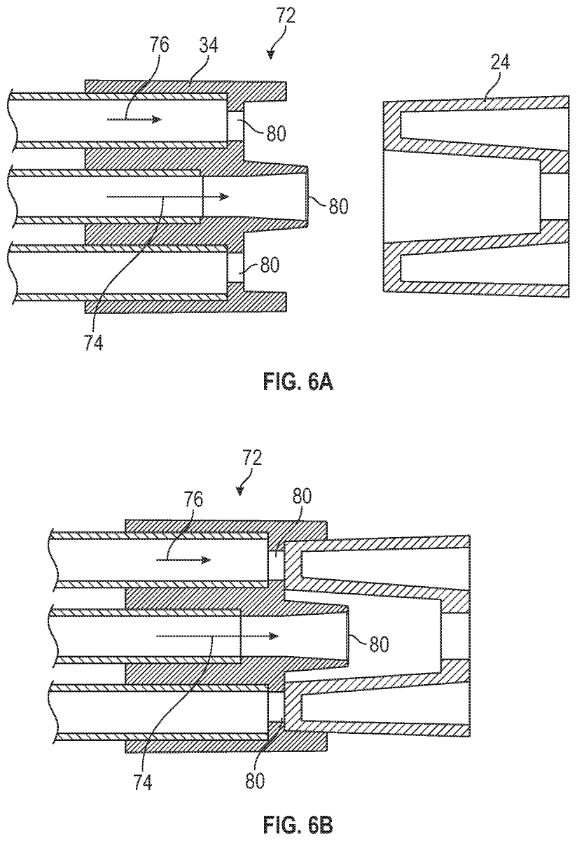

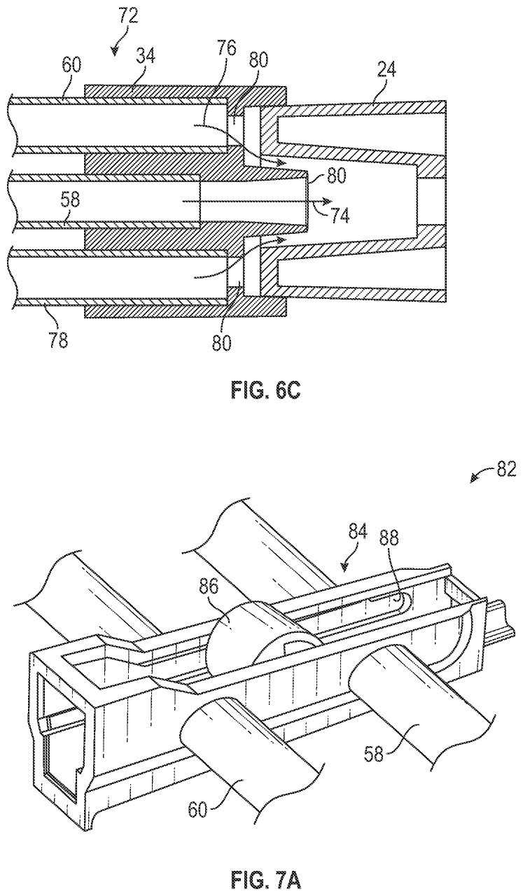

Referring now to FIGS. 6A-6C, a portion of an extension set 72 is illustrated, according to some embodiments. In some embodiments, the extension set 72 may be similar or identical to one or more of the following in terms of one or more included features and/or operation: the extension set 10 of FIGS. 1A-1C, the extension set 52 of FIGS. 2A-2C, the extension set 56 of FIGS. 3A-3C, and the extension set 70 of FIGS. 4A-4C.

In some embodiments, in response to the second luer adapter 24 being tightened with respect to the third luer adapter 34, a first fluid pathway 74 through the first extension tube 58 may be open, and a second fluid pathway 76 through the second extension tube 60 may be closed. In some embodiments, in response to the second luer adapter 24 being loosened with respect to the third luer adapter 34, the first fluid pathway 74 through the first extension tube 58 may be open, and the second fluid pathway 76 through the second extension tube 60 may be open. In some embodiments, to decrease the flow resistance within the extension set 72 after the blood collection container is partially filled and the pressure differential has decreased, the clinician may loosen the second luer adapter 24 with respect to the third luer adapter 34 or move the second luer adapter 24 from a tightened position to a loosened position. In some embodiments, the second luer adapter 24 and the third luer adapter 34 may include corresponding threads, and the second luer adapter 24 may be unthreaded with respect to the third luer adapter 34 to move the second luer adapter 24 from the tightened position to the loosened position.

In some embodiments, the extension set 72 may include a third extension tube 78, which may be integrated with the third luer adapter 34. In some embodiments, the third luer adapter 34 may include one or more openings 80, each in fluid communication with a particular extension tube. In some embodiments, the opening 80 in fluid communication with the first extension tube 58 may remain open in response to the second luer adapter 24 being in the loosened position and the tightened position with respect to the third luer adapter 34.

In some embodiments, the openings 80 in fluid communication with the second extension tube 60 and/or the third extension tube 78 may be blocked by the second luer adapter 24 when the second luer adapter 24 is in the tightened position with respect to the third luer adapter 34, as illustrated, for example, in FIG. 6B. In some embodiments, the openings 80 in fluid communication with the second extension tube 60 and/or the third extension tube 78 may be unblocked in response to the second luer adapter 24 being in the loosened position with respect to the third luer adapter 34, as illustrated, for example, in FIG. 6C. In these embodiments, the second luer adapter 24 may be spaced apart from the openings 80 in fluid communication with the second extension tube 60 and/or the third extension tube 78.

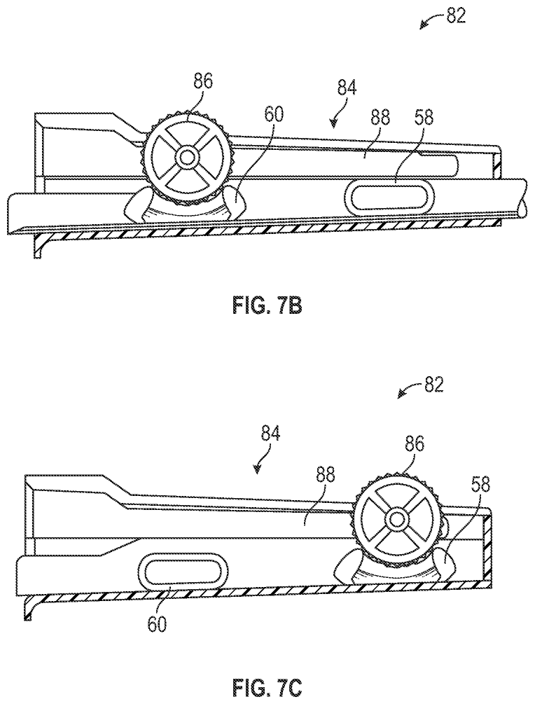

Referring now to FIGS. 7A-7C, a portion of an extension set 82 is illustrated, according to some embodiments. In some embodiments, the extension set 82 may be similar or identical to one or more of the following in terms of one or more included features and/or operation: the extension set 10 of FIGS. 1A-1C, the extension set 52 of FIGS. 2A-2C, the extension set 56 of FIGS. 3A-3C, the extension set 70 of FIGS. 4A-4C, and the extension set 72 of FIGS. 6A-6C.

In some embodiments, the extension set 82 may include a roller clamp 84. In some embodiments, the roller clamp 84 may include a roller 86 moveable within a track 88. In some embodiments, the first extension tube 58 and the second extension tube 60 may extend through the roller clamp 84 generally perpendicular to the track 88. In some embodiments, when blood flow through the second extension tube 60 is blocked, the roller 86 may be disposed in a first position proximate the second extension tube 60, as illustrated, for example, in FIG. 7B, and may pinch the second extension tube 60. In some embodiments, opening the second extension tube 60 may include moving the roller 86 along the track 88 away from the second extension tube 60 and toward the first extension tube 58. In some embodiments, when blood flow through the first extension tube 58 is blocked, the roller 86 may be disposed in a second position proximate the first extension tube 58, as illustrated, for example, in FIG. 7C, and may pinch the first extension tube 58.

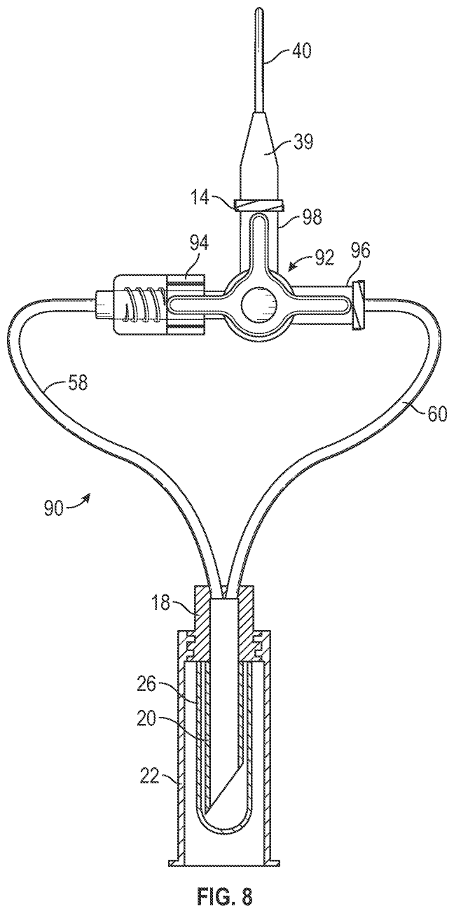

Referring now to FIG. 8, an extension set 90 is illustrated, according to some embodiments. In some embodiments, the extension set 90 may be similar or identical to one or more of the following in terms of one or more included features and/or operation: the extension set 10 of FIGS. 1A-1C, the extension set 52 of FIGS. 2A-2C, the extension set 56 of FIGS. 3A-3C, the extension set 70 of FIGS. 4A-4C, the extension set 72 of FIGS. 6A-6C, and the extension set 82 of FIGS. 7A-7C.

In some embodiments, the distal end 12 of the extension set 90 may include a three-way stopcock 92, which may include a first port 94, a second port 96, and a third port 98. In some embodiments, the first extension tube 58 may be coupled with the first port 94, the second extension tube 60 may be coupled to the second port 96, and the third port 98 may include the first luer adapter 14 configured to couple to the catheter adapter 39. In some embodiments, blocking blood flow through the second extension tube 60 may include rotating the three-way stopcock 92 to a first position. In some embodiments, opening the second extension tube 60 may include rotating the three-way stopcock 92 to a second position.

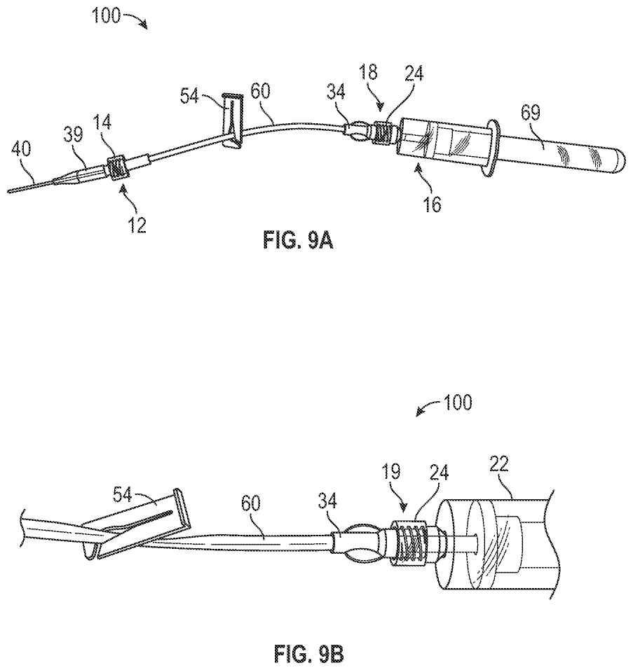

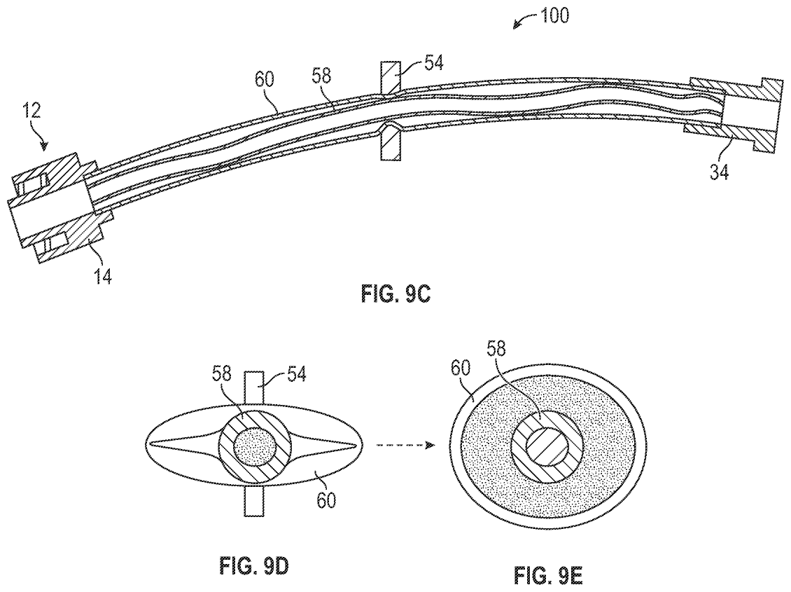

Referring now to FIGS. 9A-9E, an extension set 100 is illustrated, according to some embodiments. In some embodiments, the extension set 100 may be similar or identical to one or more of the following in terms of one or more included features and/or operation: the extension set 10 of FIGS. 1A-1C, the extension set 52 of FIGS. 2A-2C, the extension set 56 of FIGS. 3A-3C, the extension set 70 of FIGS. 4A-4C, the extension set 72 of FIGS. 6A-6C, the extension set 82 of FIGS. 7A-7C, and the extension set 90 of FIG. 8.

In some embodiments, the first extension tube 58 may be disposed within the second extension tube 60. In some embodiments, in response to the clamp 54 being in the clamped position, blood may flow through the first extension tube 58, and blood flow between an outer surface of the first extension tube 58 and an inner surface of the second extension tube 60 may decrease, as illustrated, for example, in FIG. 9D. In some embodiments, to increase the flow resistance within the catheter system after the blood collection container 69 is partially filled and the pressure differential has decreased, the clinician may move the clamp 54 to the unclamped position such that blood flows between the outer surface of the first extension tube 58 and the inner surface of the second extension tube 60, as illustrated, for example, in FIG. 9E. In some embodiments, the first extension tube 58 may be longer than the second extension tube 60 and/or an inner diameter of the first extension tube 58 may be less than an inner diameter of the second extension tube 60.

In some embodiments, the first extension tube 58 may be configured to collapse at a different pressure differential than the second extension tube 60. In some embodiments, the second extension tube 60 may include a lower durometer than the first extension tube 58. In some embodiments, the second extension tube 60 may be configured to collapse at a lower pressure differential than the first extension tube 58. In some embodiments, at the lower pressure differential, the second extension tube 60 may contact the first extension tube 58 to reduce or stop blood flow between the outer surface of the first extension tube 58 and the inner surface of the second extension tube 60. In some embodiments, in response to the blood collection container 18 partially filling with blood, the blood flow between the outer surface of the first extension tube 58 and the inner surface of the second extension tube 60 may increase.

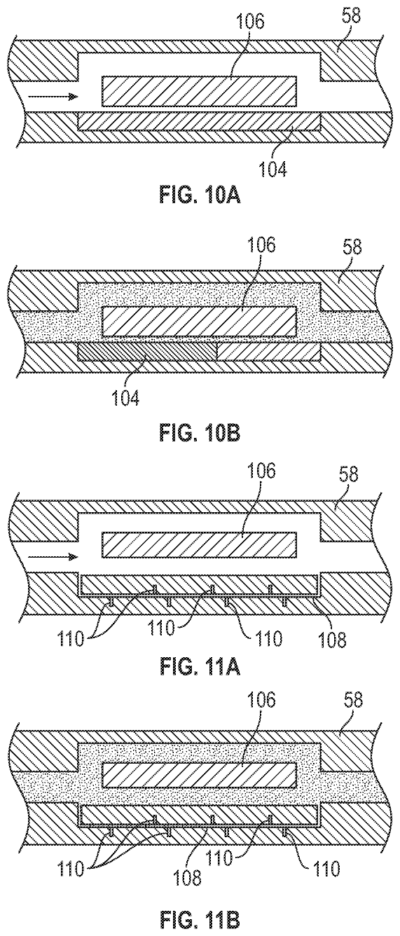

Referring now to FIGS. 10A-10B, in some embodiments, an inner surface of the first extension tube 58 may include an absorbent material 104. In some embodiments, blood within the first extension tube 58 may be configured to flow proximate the absorbent material 104. In some embodiments, in response to blood reaching or saturating a particular portion of the absorbent material 104, as illustrated, for example in FIG. 10B, or an entirety of the absorbent material 104, the clinician may change the flow resistance via a particular extension set. In some embodiments, the particular extension set may be similar or identical to one or more of the following in terms of one or more included features and/or operation: the extension set 10 of FIGS. 1A-1C, the extension set 52 of FIGS. 2A-2C, the extension set 56 of FIGS. 3A-3C, the extension set 70 of FIGS. 4A-4C, the extension set 72 of FIGS. 6A-6C, the extension set 82 of FIGS. 7A-7C, and the extension set 90 of FIG. 8.

In some embodiments, the first fluid pathway 74 through the first extension tube 58 may be narrowed proximate the absorbent material 104, such as through an island 106, which may be disposed within the first fluid pathway 74. In some embodiments, blood may flow around the island 106 in a distal to proximal direction, as illustrated, for example, in FIG. 10B.

Referring now to FIGS. 11A-11B, in some embodiments, the first extension tube 58 may include an indicator pathway or tube 108, which may include a first end and a second end connected to a primary portion of the first fluid pathway 74 of the first extension tube 58. In some embodiments, blood within the first extension tube 58 may be configured to flow through the indicator tube 108, which may include a smaller diameter than the primary portion of the first fluid pathway 74.

In some embodiments, the indicator tube 108 may include one or more markers 110, which may include grooves configured to collect blood or lines. In some embodiments, the markers 110 may provide a visual indicator to the clinician. In some embodiments, in response to blood reaching a particular marker 110, as illustrated, for example in FIG. 11B, or all of the markers 110, the clinician may change the flow resistance via a particular extension set. In some embodiments, the particular extension set may be similar or identical to one or more of the following in terms of one or more included features and/or operation: the extension set 10 of FIGS. 1A-1C, the extension set 52 of FIGS. 2A-2C, the extension set 56 of FIGS. 3A-3C, the extension set 70 of FIGS. 4A-4C, the extension set 72 of FIGS. 6A-6C, the extension set 82 of FIGS. 7A-7C, and the extension set 90 of FIG. 8.

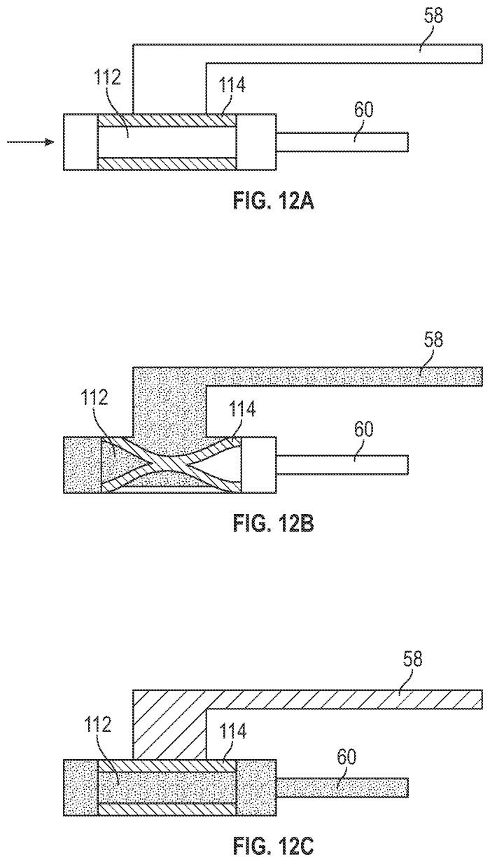

Referring now to FIGS. 12A-12C, in some embodiments, the first extension tube 58 and the second extension tube 60 may join at a junction 112. In some embodiments, the junction 112 may be disposed within the first luer adapter 14, proximal to the first luer adapter 14, or at another location within a particular extension set. In some embodiments, the particular extension set may be similar or identical to one or more of the following in terms of one or more included features and/or operation: the extension set 10 of FIGS. 1A-1C, the extension set 52 of FIGS. 2A-2C, the extension set 56 of FIGS. 3A-3C, the extension set 70 of FIGS. 4A-4C, the extension set 72 of FIGS. 6A-6C, the extension set 82 of FIGS. 7A-7C, and the extension set 90 of FIG. 8.

In some embodiments, the particular extension set may include a pressure-sensitive valve 114. In some embodiments, the pressure-sensitive valve 114 may be disposed at a junction of the first fluid pathway 74 and the second fluid pathway 76. In some embodiments, the pres sure-sensitive valve 114 may be disposed in the second extension tube 60. In some embodiments, the pressure-sensitive valve 114 may be disposed in a portion of the fluid pathway of the particular extension set distal to the first extension tube 58 and the second extension tube 60. In some embodiments, the fluid pathway of the particular extension set may extend through the proximal end 16 and the distal end 12. In some embodiments, the fluid pathway of the particular extension set may include the first fluid pathway 74 and/or the second fluid pathway 76.

In some embodiments, in response to a first predetermined pressure differential within the particular extension set, the pressure-sensitive valve 114 may be open with respect to the first extension tube 58 and closed with respect to the second extension tube 60. In some embodiments, in response to a second predetermined pressure differential within the particular extension set, the pressure-sensitive valve 114 may remain open with respect to the first extension tube 58 and open with respect to the second extension tube 60. In some embodiments, the first predetermined pressure differential may be greater than the second predetermined pressure differential. In some embodiments, the first predetermined pressure differential and the second predetermined pressure differential may correspond to the pressure differential between the vein of the patient and the blood collection container coupled to the particular extension set. In some embodiments, the pressure-sensitive valve 114 may include any suitable type of pressure-sensitive valve. In some embodiments, an inner surface of the particular extension set may include one or more grooves proximate the pressure-sensitive valve. In some embodiments, the grooves may allow blood to bypass the pressure-sensitive valve 114 and flow into the first extension tube 58 but not the second extension tube 60.

In some embodiments, a method of blood collection or collecting a blood sample may include inserting a catheter of a catheter system into vasculature of the patient. In some embodiments, the catheter system may include a catheter assembly, which may include a catheter adapter and the catheter. In some embodiments, the catheter may be secured within the catheter adapter and may extend distally from the catheter adapter.

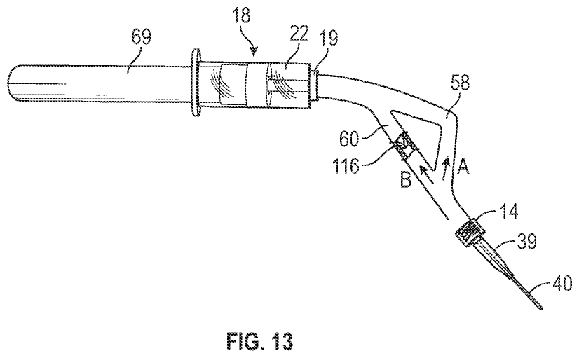

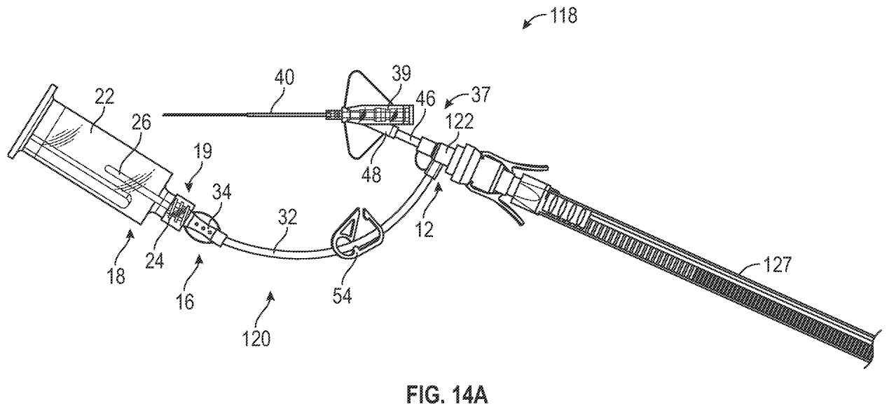

Referring now to FIG. 13, in some embodiments, a pressure-sensitive valve 116 may be disposed within the second extension tube 60, which may be shorter than the first extension tube 58 and/or include a larger inner diameter than the first extension tube 58. In some embodiments, the pressure-sensitive valve 116 may include a duckbill valve, as illustrated, for example, in FIG. 13, or another suitable type of pressure-sensitive valve. For example, the pressure-sensitive valve 116 may include a spring-loaded plunger or ball. In some embodiments, the second extension tube 60 may be more compliant than the first extension tube 58 and thus may close at a lower pressure differential than the first extension tube 58.

In some embodiments, in response to a first predetermined pressure differential within a particular extension set, the pressure-sensitive valve 116 may be closed and not allow blood to flow through the second extension tube 60. In some embodiments, in response to a second predetermined pressure differential within the particular extension set, the pressure-sensitive valve 116 may open. In some embodiments, the first predetermined pressure differential may be greater than the second predetermined pressure differential. In some embodiments, the particular extension set may be similar or identical to one or more of the following in terms of one or more included features and/or operation: the extension set 10 of FIGS. 1A-1C, the extension set 52 of FIGS. 2A-2C, the extension set 56 of FIGS. 3A-3C, the extension set 70 of FIGS. 4A-4C, the extension set 72 of FIGS. 6A-6C, the extension set 82 of FIGS. 7A-7C, and the extension set 90 of FIG. 8.

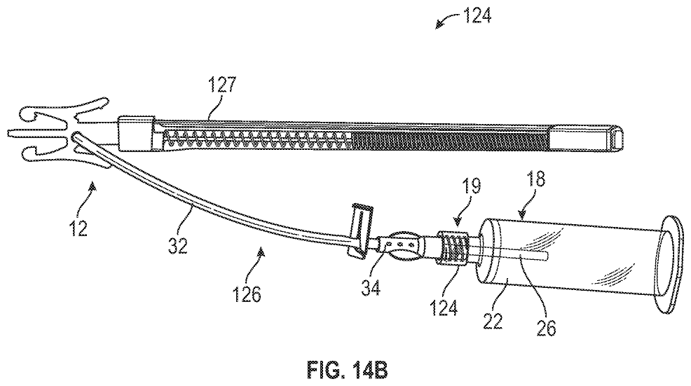

Referring now to FIG. 14A, a catheter system 118 is illustrated, according to some embodiments. In some embodiments, the catheter system 118 may include an extension set 120, which may be integrated with the catheter assembly 37. In further detail, in some embodiments, the distal end 12 of the extension set 120 may be integrated with an adapter 122 of the catheter assembly 37, as illustrated, for example, in FIG. 14A, or integrated with the catheter adapter 39 itself. In these and other embodiments, the extension set 120 may not be removable from the catheter system 118. In some embodiments, the distal end of the first extension tube 58 or the distal end of the extension tube 32 may be integrated with the adapter 122 or the catheter adapter 39. In some embodiments, the adapter 122 may include a Y-adapter, a T-adapter, or another suitable adapter.

In some embodiments, the first extension tube 58 and/or the second extension tube 60 (see, for example, FIGS. 4-9) may be integrated into the side port 48. In some embodiments, the other extension tube 46 may be similar or identical to the first extension tube 58 in terms of one or more included features and/or operation. In some embodiments, the extension set 120 may be similar or identical to one or more of the following in terms of one or more included features and/or operation: the extension set 10 of FIGS. 1A-1C, the extension set 52 of FIGS. 2A-2C, the extension set 56 of FIGS. 3A-3C, the extension set 70 of FIGS. 4A-4C, the extension set 72 of FIGS. 6A-6C, the extension set 82 of FIGS. 7A-7C, and the extension set 90 of FIG. 8. In some embodiments, the extension set 120 may be similar or identical to one or more of the following in terms of one or more included features and/or operation: the extension sets of FIGS. 9-13.

Referring now to FIG. 14B, a catheter system 124 is illustrated, according to some embodiments. In some embodiments, the catheter system 124 may include an extension set 126, which may be coupled to or integrated with an instrument delivery device 127, which may deliver a probe, a catheter, or a guidewire through a particular catheter assembly (as illustrated, for example, in FIG. 14A). In some embodiments, the first extension tube 58 and/or the second extension tube 60 (see, for example, FIGS. 4-9) may be integrated into the instrument delivery device 127.

In some embodiments, the extension set 126 may be similar or identical to one or more of the following in terms of one or more included features and/or operation: the extension set 10 of FIGS. 1A-1C, the extension set 52 of FIGS. 2A-2C, the extension set 56 of FIGS. 3A-3C, the extension set 70 of FIGS. 4A-4C, the extension set 72 of FIGS. 6A-6C, the extension set 82 of FIGS. 7A-7C, and the extension set 90 of FIG. 8. In some embodiments, the extension set 126 may be similar or identical to one or more of the following in terms of one or more included features and/or operation: the extension sets of FIGS. 9-13.

In some embodiments, the instrument delivery device 127 may include any suitable instrument delivery device. In some embodiments, the instrument delivery device 127 may be further described in U.S. patent application Ser. No. 16/037,246, filed Jul. 17, 2018, entitled “EXTENSION HOUSING A PROBE OR INTRAVENOUS CATHETER,” U.S. patent application Ser. No. 16/388,650, filed Apr. 18, 2019, entitled “INSTRUMENT DELIVERY DEVICE HAVING A ROTARY ELEMENT,” U.S. patent application Ser. No. 16/037,319, filed Jul. 17, 2018, entitled “MULTI-DIAMETER CATHETER AND RELATED DEVICES AND METHODS,” U.S. patent application Ser. No. 16/502,541, filed Jul. 3, 2019, entitled “DELIVERY DEVICE FOR A VASCULAR ACCESS INSTRUMENT,” U.S. patent application Ser. No. 16/691,217, filed Nov. 21, 2019, entitled “SYRINGE-BASED DELIVERY DEVICE FOR A VASCULAR ACCESS INSTRUMENT,” U.S. Patent Application No. 62/794,437, filed Jan. 18, 2019, entitled “CATHETER DELIVERY DEVICE AND RELATED SYSTEMS AND METHODS,” and U.S. Patent Application No. 62/830,286, filed Apr. 5, 2019, entitled “VASCULAR ACCESS INSTRUMENT HAVING A FLUID PERMEABLE STRUCTURE AND RELATED DEVICES AND METHODS,” which are incorporated by reference in their entirety.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

We claim:

1. A catheter extension set, comprising:
a distal end, comprising a luer adapter configured to couple to a catheter adapter;
a proximal end, comprising a blood collection device; and
an extension tube extending between the distal end and the proximal end.

2. The catheter extension set of claim 1, further comprising a clamp disposed on the extension tube, wherein the clamp is configured to move between a clamped position and an unclamped position.

3. The catheter extension set of claim 1, wherein the luer adapter is a first luer adapter, wherein the blood collection device comprises a second luer adapter, further comprising a third luer adapter coupled to the second luer adapter, wherein the extension tube comprises a distal end and a proximal end, wherein the distal end of the extension tube is integrated with the first luer adapter, wherein the proximal end of the extension tube is integrated with the third luer adapter.

4. The catheter extension set of claim 1, wherein the extension tube comprises a distal end and a proximal end, wherein the distal end of the extension tube is integrated with the luer adapter, wherein the proximal end of the extension tube is integrated with the blood collection device.

5. The catheter extension set of claim 1, wherein an inner surface of the extension tube comprises an absorbent material or an indicator tube comprising a plurality of markers, wherein blood within the extension tube is configured to flow proximate the absorbent material or through the indicator tube.

6. A catheter extension set, comprising:
a distal end, comprising a luer adapter configured to couple to a catheter adapter;
a proximal end, comprising a blood collection device;
a first extension tube extending between the distal end and the proximal end; and
a second extension tube extending between the distal end and the proximal end.

7. The catheter extension set of claim 6, further comprising a clamp, wherein the second extension tube extends through the clamp, wherein the clamp is configured to move between a clamped position and an unclamped position.

8. The catheter extension set of claim 7, wherein the first extension tube is longer than the second extension tube.

9. The catheter extension set of claim 7, wherein an inner diameter of the first extension tube is less than an inner diameter of the second extension tube.

10. The catheter extension set of claim 7, wherein an inner surface of the first extension tube comprises an absorbent material or an indicator tube comprising a plurality of markers, wherein blood within the extension tube is configured to flow proximate the absorbent material or through the indicator tube.

11. The catheter extension set of claim 7, wherein the first extension tube is disposed within the second extension tube, wherein in response to the clamp being in the clamped position, blood flows through the first extension tube and blood flow between an outer surface of the first extension tube and an inner surface of the second extension tube.

12. The catheter extension set of claim 5, wherein the luer adapter is a first luer adapter, wherein the blood collection device comprises a second luer adapter, further comprising a third luer adapter coupled to the second luer adapter, wherein the first extension tube comprises a distal end and a proximal end, wherein the second extension tube comprises a distal end and a proximal end, wherein the distal end of the first extension tube and the distal end of the second extension tube are integrated with the first luer adapter, wherein the proximal end of the first extension tube and the proximal end of the second extension tube are integrated with the third luer adapter, wherein in response to the second luer adapter being tightened with respect to the third luer adapter, a first fluid pathway through the first extension tube is open and a second fluid pathway through the second extension tube is closed, wherein in response to the second luer adapter being loosened with respect to the third luer adapter, the first fluid pathway through the first extension tube is open and the second fluid pathway through the second extension tube is open.

13. The catheter extension set of claim 5, further comprising a pressure-sensitive valve within a fluid pathway of the catheter extension distal to the first extension tube and the second extension tube, wherein in response to a first predetermined vacuum pressure level within the catheter extension set, the pressure-sensitive valve is open with respect to the first extension tube and closed with respect to the second extension tube, wherein in response to a second predetermined vacuum pressure level within the catheter extension set, the pressure-sensitive valve is closed with respect to the first extension tube and open with respect to the second extension tube, wherein the first predetermined vacuum pressure level is greater than the second predetermined vacuum pressure level.

14. The catheter extension set of claim 5, wherein the second extension tube comprises a pressure-sensitive valve.

15. A method of blood collection, comprising:
inserting a catheter of a catheter system into vasculature of a patient, wherein the catheter system comprises:
a catheter adapter;
the catheter extending distally from the catheter adapter;
coupling a catheter extension set to the catheter adapter, wherein the catheter extension set comprises:
a distal end, comprising a luer adapter configured to couple to a catheter adapter;
a proximal end, comprising a blood collection device, wherein the blood collection device comprises a needle configured to receive a blood collection container;
a first extension tube extending between the distal end and the proximal end; and
a second extension tube extending between the distal end and the proximal end;
blocking blood flow through the second extension tube;
after inserting the catheter of the catheter system into the vasculature of the patient, coupling the catheter extension set to the catheter adapter, and blocking blood flow through the second extension tube, coupling a blood collection container to the blood collection device, wherein in response to coupling the blood collection container to the blood collection device, the blood collection container begins to fill with blood;
in response to the blood collection container filling partially with blood, opening the second extension tube such that increased blood flows through the second extension tube.

16. The method of claim 15, wherein a flow resistance of the first extension tube is greater than a flow resistance of the second extension tube.

17. The method of claim 15, wherein the catheter system further comprises a clamp, wherein the second extension tube extends through the clamp, wherein the clamp is configured to move between a clamped position and an unclamped position, wherein blocking blood flow through the second extension tube comprises moving the clamp to the clamped position, wherein opening the second extension tube comprises moving the clamp to the unclamped position.

18. The method of claim 15, wherein the catheter system further comprises a roller clamp, wherein the roller clamp comprises a roller moveable within a track, wherein the first extension tube and the second extension tube extend through the roller clamp generally perpendicular to the track, wherein when blood flow through the second extension tube is blocked, the roller is disposed in a first position proximate the second extension tube, wherein opening the second extension tube comprises moving the roller along the track away from the second extension tube and toward the first extension tube.

19. The method of claim 15, wherein the luer adapter is a first luer adapter, wherein the blood collection device comprises a second luer adapter, further comprising a third luer adapter coupled to the second luer adapter, wherein the first extension tube comprises a distal end and a proximal end, wherein the second extension tube comprises a distal end and a proximal end, wherein the distal end of the first extension tube and the distal end of the second extension tube are integrated with the first luer adapter, wherein the proximal end of the first extension tube and the proximal end of the second extension tube are integrated with the third luer adapter, wherein blocking blood flow through the second extension tube comprises tightening the second luer with respect to the third luer adapter, wherein opening the second extension tube comprises loosening the second luer with respect to the third luer adapter.

20. The method of claim 15, wherein the distal end of the catheter extension set comprises a three-way stopcock having a first port, a second port, and a third port, wherein the first extension tube is coupled with the first port, the second extension tube is coupled to the second port, and the third port comprises the luer adapter configured to couple to the catheter adapter, wherein blocking blood flow through the second extension tube comprises rotating the three-way stopcock to a first position, wherein opening the second extension tube comprises rotating the three-way stopcock to a second position.

21. A catheter extension set, comprising:
a distal end, comprising a luer adapter configured to couple to a catheter adapter;
a proximal end, comprising another luer adapter configured to couple to a blood collection device; and
an extension tube extending between the distal end and the proximal end, wherein a geometric factor G_{f} of the extension tube is equal to or greater than a predetermined value to reduce hemolysis.

22. The catheter extension set of claim 21, wherein the catheter extension set is configured for use with an 18G catheter, wherein the geometric factor G_{f} of the extension tube is equal to or more than 3.83E+06 (l/in³).

23. The catheter extension set of claim 21, wherein the catheter extension set is configured for use with an 20G catheter, wherein the geometric factor G_{f} of the extension tube is equal to or more than 3.27E+06 (l/in³).

24. The catheter extension set of claim 21, wherein the catheter extension set is configured for use with an 22G catheter, wherein the geometric factor G_{f} of the extension tube is equal to or more than 3.33E+06 (l/in³).

25. The catheter extension set of claim 21, wherein the catheter extension set is configured for use with an 24G catheter, wherein the geometric factor G_{f} of the extension tube is equal to or more than 1.50E+07 (l/in³).
